(19) United States
(12) Patent Application Publication
(10) Pub. No.: US 2007/0207487 A1
(43) Pub. Date: Sep. 6, 2007

(54) Photoelectrochemical synthesis of high density combinatorial polymer arrays

(76) Inventors: Christopher J. Emig, Napa, CA (US); Brian Y. Chow, Cambridge, MA (US); Joseph M. Jacobson, Newton, MA (US)

Correspondence Address: NORMA E HENDERSON;HENDERSON PATENT LAW, 13 JEFFERSON DR, LONDONDERRY, NH, 03053, US

(21) Appl. No.: 11/698,282

(22) Filed: Jan. 25, 2007

Related U.S. Application Data
(60) Provisional application No. 60/761,844, filed on Jan. 25, 2006.

(51) Int. Cl. C12Q 1/68; C12M 3/00; H01L 21/00

(52) U.S. Cl. 435/6; 435/287.2; 438/1

(57) ABSTRACT

In a method for creating polymer arrays through photoelectrochemically modulated acid/base/radical generation for combinatorial synthesis, electrochemical synthesis is guided by a spatially modulated light source striking a semiconductor in an electrolyte solution. A substrate having at its surface at least one photoelectrode that is proximate to at least one molecule bearing at least one chemical functional group is provided, along with a reagent-generating chemistry co-localized with the chemical functional group and capable of generating reagents when subjected to a potential above a threshold. An input potential is then applied to the photoelectrode that exceeds the threshold in the presence of light and that does not exceed the threshold in the absence of light, causing the transfer of electrons to or from the substrate, and creating a patterned substrate. The process is repeated until a polymer array of desired size is created.

### RELATED APPLICATIONS

This application claims the benefit of U.S. Provisional Application Ser. No. 60/761,844, filed Jan. 25, 2006, the entire disclosure of which is herein incorporated by reference.

### STATEMENT REGARDING FEDERALLY SPONSORED RESEARCH OR DEVELOPMENT

This invention was made with U.S. government support under Grant Number CCR-0122419, awarded by the National Science Foundation. The government has certain rights in this invention.

### FIELD OF THE INVENTION

This invention relates to creation of polymer arrays and, in particular, to generation of polymer arrays by photoelectrochemical patterning.

### BACKGROUND

In recent years, hybridization arrays [e.g., Fodor S P, R. J., Pirrung M C, Stryer L, Lu A T, Solas D. (1991), “Light-directed, spatially addressable parallel chemical synthesis”, Science 251(4995): 767-73] and on-chip sequencing-by-synthesis [e.g., Quake, E. P. K. a. S. R. (2004), “Microfluidic device reads up to four consecutive base pairs in DNA sequencing-by-synthesis”, Nucleic Acids Research 32(9): 2873-2879] have revolutionized genome sequencing because of their massively parallel genomic sequencing power. Several techniques for in-situ DNA array synthesis [e.g., U.S. Pat. Nos. 6,054,270; 5,700,637] have been reported in the literature: photocleavable 5′ and 3′ protecting groups [Fodor (1991); U.S. Pat. Nos. 5,445,934, 5,744,305, 5,677,195; Singh-Gasson, S., R. D. Green, et al. (1999), “Maskless fabrication of light-directed oligonucleotide microarrays using a digital micromirror array.” Nature Biotechnology 17(10): 974-978], deprotection of the acid labile trityl groups by photogenerated acids (Gao, X., E. LeProust, et al. (2001), “A flexible light-directed DNA chip synthesis gated by deprotection using solution photogenerated acids”, Nucleic Acids Research 29(22): 4744-4750], and electrochemical acid generation [Dill, K., D. D. Montgomery, et al. (2004), “Immunoassays and sequence-specific DNA detection on a microchip using enzyme amplified electrochemical detection”, Journal of Biochemical and Biophysical Methods 59(2): 181-187; U.S. Pat. No. 6,093,302; Egeland, R. D. and E. M. Southern (2005), “Electrochemically directed synthesis of oligonucleotides for DNA microarray fabrication”, Nucl. Acids Res. 33(14): e125], ink-jetting reagents [Hughes, T. R., M. M., Jones A R et al. (2001), Nat. Biotech. 4:342-347], and microcontact printing [Xiao, P. F., N. Y. He, et al. (2002), “In situ synthesis of oligonucleotide arrays by using soft lithography”, Nanotechnology 13(6): 756-762].

Chips synthesized using photocleavable protecting groups have demonstrated high spot densities, but the reagent costs for such phosphoramidite reagents are currently prohibitively high for non-commercial laboratories (typically more than $300/g). The UV exposure deprotection times are also quite long, usually on the order of minutes. Methods that locally photogenerate acids to detritylate standard phosphoramidite reagents do offer improved deprotection times [Gao (2001)], but they have yet to show the same spot density as those generated using photocleavable protecting groups, presumably because of acid diffusion. Systems based on electrochemical acid generation have also demonstrated quick deprotection times, but circuitry required to make individually addressable electrodes limits is costly to fabricate by photolithographic means [e.g., U.S. Pat. No. 6,093,302]. Extremely high-resolution chips have been created by micro-contact printing of phosphoramidites, but this technique is limited in its versatility because it requires the creation of a new stamp for each base added [Xiao (2002)].

A low-cost and rapid synthesis platform of high-resolution DNA chips would be of great utility to laboratory scientists. Such a platform would open up many other exciting possibilities in biological research; for example, it has been recently shown that genomic length DNA can be assembled from the short oligonucleotides from such chips. Furthermore, the techniques used to create DNA chips can be used to pattern other biomolecules [Shuwei Li, D. B., Nishanth Marthandan, Stanley Klyza, Kevin J. Luebke, Harold R. Garner, and Thomas Kodadek (2004), “Photolithographic Synthesis of Peptoids”, J. Am. Chem. Soc. 126(13): 4088 -4089] and polymers on surfaces. What has been needed, therefore, is a means for quickly and inexpensively creating polymer arrays.

### SUMMARY

The present invention is a method for creating inexpensive oligonucleotide, protein, or other polymer arrays through photoelectrochemically modulated acid/base/radical generation for combinatorial synthesis, where electrochemical synthesis is guided by a spatially modulated light source striking a semiconductor in an electrolyte solution. A semiconducting device is in contact with an electrolyte solution, matrix, gel, or solid that is suitable as a platform for electrochemical reactions at a surface. Light patterned by a mask, LED, LCD, steered mirror, or digital micromirror array is used to generate charge carriers in the semiconductor, which then generate electrochemical reactions via direct transfer of electrons to or from the semiconductor, optionally through a metal or mediator, thus allowing for spatially-guided electrochemistry.

In one aspect, the present invention is a method to immobilize and/or in-situ build biomolecule, bio-polymer, small molecule, and polymer arrays by photoelectrochemical patterning. In particular, the present invention may be employed to fabricate DNA arrays by phosphoramidite synthesis.

In another aspect, the present invention is a method for photoelectrochemical placement of a material at a specific location on a substrate. A substrate having at its surface at least one photoelectrode that is proximate to at least one molecule bearing at least one chemical functional group is provided, along with a reagent-generating chemistry co-localized with the chemical function group and capable of generating reagents when subjected to a potential above a threshold. An input potential is then applied to the photoelectrode that exceeds the threshold in the presence of light and that does not exceed the threshold in the absence of light, causing the transfer of electrons to or from the substrate. In one embodiment, the chemical functional group is protected and the generated reagents are deprotecting. In another embodiment, the chemical functional group is unprotected and the generated reagents are activating. In a further embodiment, the protected chemical functional group is located on a second parallel substrate and the reagent-generating chemistry can diffuse towards the protected chemical function group on the second substrate.

In a further aspect, the present invention is a method for photoelectrochemical synthesis of a polymer array, comprising the steps of providing a substrate having at the substrate surface at least one photoelectrode that is proximate to at least one molecule bearing at least one chemical functional group, providing a reagent-generating chemistry co-localized with the chemical function group capable of generating reagents when subjected to a potential above a threshold, applying an input potential to the photoelectrode that exceeds the threshold in the presence of light and that does not exceed the threshold in the absence of light, and repeating until a polymer array of desired size is created.

### BRIEF DESCRIPTION OF THE DRAWINGS

The foregoing discussion will be understood more readily from the following detailed description of the invention, when taken in conjunction with the accompanying drawings, in which like referenced features identify common features in corresponding drawings and:

FIG. 1 is a schematic of an embodiment of a system employed for photoelectrochemical synthesis of polymer arrays, according to one aspect of the present invention;

FIG. 2 illustrates the steps in two embodiments of a method for the photoelectrochemical placement of a material at a specific location on a substrate to create a photoelectrode substrate, according to one aspect of the present invention;

FIG. 3 is an Scanning Electron Microscope micrograph of patterned platinum microcontacts on amorphous silicon over ITO-glass, as created according to one aspect of the present invention;

FIGS. 4A and 4B are Atomic Force Microscopy and Scanning Electron Microscope micrographs, respectively, of a porous SiO2 reaction layer created from sintered commercial colloidal silica, according to one aspect of the present invention;

FIG. 5 is a schematic of an embodiment of a DMD-based spatially modulated illumination system used to drive site-selective photoelectrochemistry, according to one aspect of the present invention;

FIG. 6 is a photoelectrochemical cyclic voltammogram of ferrocene demonstrating selective photo-induced redox chemistry; and

FIG. 7 is a fluorescence micrograph of a dye that has selectively reacted with functional groups that are only photoelectrochemically deprotected over illuminated electrodes, according to one aspect of the present invention.

### DETAILED DESCRIPTION

The present invention is a method and apparatus for creating inexpensive nucleotide, protein, or other polymer arrays through photoelectrochemically modulated acid/base/radical generation for combinatorial synthesis, where electrochemical synthesis is guided by a spatially modulated light source striking a semiconductor in an electrolyte solution. A semiconducting device is in contact with an electrolyte solution, matrix, gel, or solid suitable as a platform for electrochemical reactions at a surface. Light patterned by a mask, LED, LCD, steered mirror or digital micromirror array is used to generate charge carriers in the semiconductor, which then generate electrochemical reactions via direct transfer of electrons to or from the semiconductor, or through a metal or mediator, thus allowing for spatially guided electrochemistry. As all addressing occurs off of the device, one skilled in the art can recognize and appreciate that substrate costs are minimal.

In one aspect, the present invention comprises a method to immobilize and/or in-situ build biomolecule, bio-polymer, small molecule, and polymer arrays by photoelectrochemical patterning. In particular, the present invention permits the fabrication of DNA arrays by phosphoramidite synthesis. This technique has been used to pattern and move biomolecules and polymer beads, to move fluids in microfluidics by opto-electrowetting (Chiou, P. Y., H. Moon, et al. (2003), “Light actuation of liquid by optoelectrowetting.” Sensors and Actuators, A: Physical A104(3): 222-228), and to control electrochemistry for analyte detection (Hafeman, et al. (1988), “Light-Addressable Potentiometric Sensor for Biochemical Systems”; George, M., W. J. Parak, et al. (2000), “Investigation of the spatial resolution of the light-addressable potentiometric sensor”, Sensors and Actuators A: Physical 86(3): 187-196; Tatsu Yoshinobu, et al. (2004), “Fabrication of-Thin-Film LAPS with Amorphous Silicon”; U.S. Pat. No. 6,682,648). Because the electrodes are light addressable, there are no physically addressable electrodes required, and thus the fabrication of such a device is cheap and facile, while still offering high resolution or spot density. Reagent costs are low as the platform relies on standard phosphoramidite reagents. The present invention therefore allows DNA and polymer arrays to be manufactured more cheaply and faster than with current alternative methods.

In a typical embodiment, the device is held in a fluidic capable of electrochemical synthesis and chemical resistance to solvents, acids, and bases. Nickel or nickel alloys, glass, fluorinated hydrocarbons (such as Teflon), fluorinated elastomers, high density polyethylene or polypropylene, gold, and platinum group metals (PGMs) are examples of suitable materials, but other materials may also be advantageously employed in the present invention. Teflon, glass, and platinum are preferred for their inertness to electrochemical processes. The fluidic must support the connections to the semiconducting device electrode and maintain the counter electrode in contact with the electrolyte. The fluidic must also have a window to the semiconductor surface, through which the semiconductor is illuminated. This window may contact the solution (“front side” illumination) so that light illuminates the semiconductor through solution. It is preferable that the window also be a conductor, so that the surface area of the counter electrode is at least as large as that of the semiconducting electrode surface. A clear conducting oxide is an example of a suitable material, and is even better if a thin passivation layer of platinum, palladium, or iridium is applied to the surface such that it still transmits enough light to the semiconducting surface. If the window enables front-side illumination, it is possible to directly monitor the removal of the protecting group or the generation of the protecting reagent in real-time. For example, UV-Vis absorbance measurements or imaging with a color-filtered CCD camera may be employed, since the trityl protecting group absorbs at ˜298 nm and a protected phosphoramidite is clear in solution, but the post-deprotection cation absorbs light at ˜498 nm and the solution turns orange. Alternatively, the window may directly contact the semiconductor (such as a glass/ITO window), which is known as “back side” illumination. In this manner, the ITO/glass serves as both a window and an electrical contact.

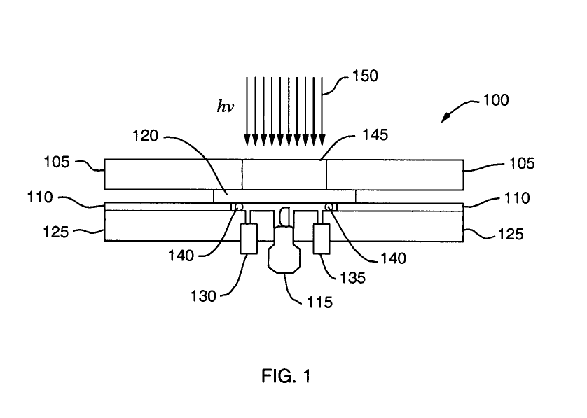

FIG. 1 is a schematic of an embodiment of a system for photoelectrochemical synthesis of polymer arrays, according to one aspect of the present invention. In the preferred embodiment of FIG. 1, the photoelectrochemistry fluidic for photoelectrochemical patterning comprises fluidic chamber 100 that includes plastic compression fittings 105, Teflon spacers 110 (glued via Viton), reference electrode 115 (platinum quasi-reference), photoelectrode substrate 120, counter electrode 125 (nickel fluidic), fluid inlet 130, fluid outlet 135, Kalrez O-rings 140, and window 145 for illumination from a light source 150 (hv). FIG. 1 depicts a “back-side” illumination fluidic, as light passes through the substrate instead of through solution. Depending on the system specifications, solution side (front side) or substrate side (back side) illumination may be preferable, in which case the device preparation and fluidic design is adjusted accordingly, as is well-known in the art of the invention.

The photoelectrode substrate is preferably a semiconductor. The semiconductor may be intrinsic, n-type, p-type, or some multi-layer structure such as a PIN photodiode operated in reverse bias. Intrinsic or n-type semiconductors, such as, but not limited to, TiO2, amorphous or crystalline silicon, zinc sulphide, and cadmium selenide, have better etch resistance properties when under a positive bias with respect to the electrolyte solution if they are to be in direct contact. In general, silicon is typically preferred, because the processing steps in passivating the surface from electrochemical or chemical degradation are easier.

The semiconductor may be covered with a protective material, such as, for example, an inert metal or a different semiconductor, in order to take advantage of the respective material properties. For example, amorphous silicon may be covered with mesoporous or flat titanium dioxide, thereby creating a surface suitable for direct, stable electrochemical reaction, but still having silicon dominate the photoconductive gain. Alternatively, for example, amorphous silicon may be covered with thin silicon nitride, which is known to prevent the dehydrogenation of amorphous silicon, thereby improving the photosensitivity and lifetime of the photoconductor. The electrolyte solution can be aqueous, organic, or ionic, depending on the semiconductor used and the desired reaction to take place.

In a typical embodiment, the semiconducting device is under the application of an electric field, either intrinsic to the device, such as via a PIN structure, or externally applied, in order to guide the charge carriers generated by illumination in a desired direction, but the selection of semiconducting material or layers of material may be such that photoexcited charge moving across the semiconductor space charge layer in contact with the solution has enough energy to react directly with an electrolyte. Upon illumination with a specific light pattern, areas under illumination will generate charge carriers, and thus be capable of electron capture from the solution to the semiconductor and overall decreased impedance of the semiconductor layer. In an alternate embodiment, upon illumination with a specific light pattern, areas under illumination will generate charge carriers, and thus be capable of electron injection to the solution from the semiconductor.

Whereas the applied bias potential drop is primarily across the semiconductor in a non-illuminated semiconductor-electrolyte interface [Bard, A. J., Stratman, M. S., and Licht, S. Semiconductor Electrodes and Photoelectrochemistry. Wiley-VCH: Chapter 1], the potential drop is nearly entirely shifted to the double layer of the electrolyte when the photoconductive electrode is illuminated with sufficient light because of the decreased impedance. Thus, non-illuminated electrodes are at much lower potentials at the interface than the bias potential, but illuminated ones are effectively at the bias potential at the interface. This provides the contrast necessary to perform a desired reaction at only the locations that are illuminated, since electrochemical reactions are highly non-linear with respect to surface potential. Features as small or smaller than a few microns are possible utilizing the present invention, demonstrating that the present invention compares with current commercially available high-density arrays, while being considerably cheaper to manufacture. In the preferred embodiment, the surface is biased at or above the activation energy or threshold voltage, but still within the potential window of the electrolyte system.

A typical device is fabricated with plasma enhanced vapor deposition (PECVD) of amorphous silicon onto a suitable conductive surface, such as indium tin oxide. A front side illuminated device may have a thin film of amorphous silicon deposited onto steel or aluminum or any other conductor with appropriate adhesion to silicon, or may be simply a wafer of crystalline silicon with an Ohmic contact, such as evaporated/annealed gold. The front side illumination system is preferable if there is low or nonexistent applied external bias or high dopant concentration, because more charge carriers are generated at the surface and thus are less likely to recombine before reacting with the electrolyte (as opposed to charge carriers generated in the bulk, which must first diffuse to a space charge region in the semiconductor). A backside-illuminated device may have a thin film (typically 500 nm to 2 um) of amorphous silicon deposited onto a clear conducting oxide, such as indium tin oxide, on a flat glass slide (such as Coming 1737 Glass or polished float glass), which serves as the window to the semiconductor. Front-side illumination systems enable the additional use of photocleavable and photogenerated chemistries. A combination of both front- and back-side illumination may also be employed.

In one embodiment, material is photoelectrochemically placed at a specific location on a substrate by providing a substrate having at the substrate surface at least one photoelectrode that is proximate to at least one molecule bearing at least one chemical functional group, providing a reagent-generating chemistry co-localized with the chemical function group capable of generating reagents when subjected to a potential above a threshold, and applying an input potential to the photoelectrode that exceeds the threshold in the presence of light and that does not exceed the threshold in the absence of light. In another embodiment, a polymer array is synthesized by providing a substrate having at the substrate surface at least one photoelectrode that is proximate to at least one molecule bearing at least one chemical functional group, providing a reagent-generating chemistry co-localized with the chemical function group capable of generating reagents when subjected to a potential above a threshold, applying an input potential to the photoelectrode that exceeds the threshold in the presence of light and that does not exceed the threshold in the absence of light, and repeating until a polymer array of desired size is created.

When the array is being constructed by selectively activating/deprotecting a site and immobilizing pre-synthesized molecules, the reactive group can sit directly atop the electrode, and direct photoelectrochemistry may be employed [e.g., Kim et al. (2002), Langmuir 18, 1460-1462]. This should typically be avoided for in-situ synthesized arrays, because electrochemical damage to the growing polymer may occur. Adequate linking chemistry is necessary that can withstand electrochemical processing, allowing for reagents to diffuse properly and adequately bind the DNA to the substrate. This linking chemistry can be broken down into two parts, with the first being the development of an insulating microporous reaction layer bound to the surface of the chip that allows the growing DNA strand to be proximal to the electrochemistry during the deprotection step but sufficiently far from the electrode (beyond the Helmholtz layer, ˜5-10 nm) to not incur electrochemical damage. The electrochemistry may also generate activating/catalytic chemistry instead of deprotecting reagents. For example, an acid created may be used to deprotect a dimethoxytrityl group during a phosphoramidite synthesis as shown, or activate the phosphoramidite addition towards an already deprotected hydroxyl.

A porous reaction layer has the benefit of vastly increasing the surface area of the chip, thereby increasing the total crude product quantity yield (U.S. Pat. No. 6,824,866). In some cases, this layer can also increase the crude product quality yield because of increased mass transport and decreased sterics (Zhou et al. Nucleic Acids Res. 2004, 32, 18, 5409-5417). Data demonstrating the increased quantity and quality from a porous reaction layer is presented in Table 1, which is a table of surface loading capacities of DNA phosphormidites and as-synthesized oligonucleotides on the porous reaction layer according to one aspect of the present invention.

TABLE 1

| | | | 40-mer oligonucleotide | | | | | |
|---|---|---|---|---|---|---|---|---|
| | Dimethoxytrityl cation | | Area density | | Concentration | | Crude | Step-wise |
| | Area density | Concentration | (molecules/cm²) | | (mM) | | yield | yield |
| Substrate | (molecules/cm²) | (mM) | Unpurified | Purified | Unpurified | Purified | (%) | (%) |
| Flat glass | 1.8 ± 0.1 × 10¹⁴ | N/A | 3.1 ± 0.5 × 10¹³ | 1.9 ± 0.3 × 10¹³ | N/A | N/A | 59.4 ± 3.4 | 98.7 ± 0.1 |
| Porous oxide | 1.3 ± 0.2 × 10¹⁶ | 25.0 ± 2.8 | 4.7 ± 0.2 × 10¹⁴ | 2.4 ± 0.2 × 10¹⁴ | 9.4 ± 0.4 | 4.7 ± 0.4 | 76.4 ± 5.3 | 99.3 ± 0.2 |
| CPG | N/A | 13.2 ± 0.3 | N/A | N/A | 7.3 ± 0.1 | 5.9 ± 0.3 | 85.5 ± 5.2 | 99.6 ± 0.2 |

The second part of the process of the present invention employs a suitable linking chemistry that binds the DNA to the microporous reaction layer. This includes, but is not limited to: alkenes for hydrosilylation to silicon, thiols and amines on metals, and organosilanes, carboxylic acids, and phosphonic acids to metals, metal oxides, semiconductors, and semiconductor oxides. It is also possible for electrochemically-generated acid that diffuses from the semiconducting substrate to react with molecules on the surface of another solid support. For example, the device can be inverted so that the reactive chemical species is in near proximity to the surface upon which DNA is synthesized, thereby allowing the device structure to be reused.

The microporous reaction layer may be composed of a wide range of materials produced by a variety of methods. Such reaction layers include, but are not limited to, porous oxidized aluminum (via chemical or electrochemical oxidation), porous silicon (via electrochemical etching), a porous titanium dioxide layer made from solution phase nanoparticles or sol-gel processing, a porous polysilicon layer, a porous silicon dioxide layer made from colloidal silica, silane sol-gels, spin-on-glasses, a porous silicon dioxide formed by post-deposition chemical or ion-etching, or the immobilization of standard solid-phase supports like controlled-pore glass (CPG) and Merrifield resins. Of these materials, silicon dioxide is preferable for the facility of silane coupling and chemical inertness, though all of the materials mentioned are capable of suitable linking chemistry for functionalization. In the case of electrochemical acid diffusion to react upon an alternative surface, a porous reaction layer is not necessary, though it may be capable of generating improved total amount of product because of the increased surface area for linking chemistry.

The surface of a DNA microarray must have a suitable linking chemistry to attach a nascent strand of DNA and withstand the rigors of the phosphoramidite synthesis cycle. Silane functionalization is a widely used technique for DNA microarrays because of their reactivity with glass surface hydroxyls and their stability. Fortunately, glass is rather easy to functionalize with organo-silane linkers, although it has been demonstrated that silanization is possible with a variety of metal oxide surfaces, including titania and alumina. Generally, a silane precursor, such as a hydroxyl or amino functionalized chlorosilane or alkoxysilane, is reacted with the free hydroxyls on an oxide surface and then permitted to cross polymerize with heat or exposure to air to form a resistant polysilane monolayer. In a typical embodiment, treatment with triethoxysilane hydroxybutyramide or 3-aminopropyl triethoxysilane provides a free hydroxyl group or amine respectively, which are then used for the attachment of further chemistries, such as, but not limited to, phosphoramidites used in DNA synthesis. These are suitable linking chemistries for surfaces with free hydroxyl groups, such as SiO2, TiO2, and most materials that have surface oxides. Alternatively, a hydrosilation reaction can be used to provide an attachment chemistry to an HF treated silicon surface, which has a hydrogen terminated surface. Suitable hydrosilation coupling compounds are generally a terminal alkene with a protected nucleophile at the opposing end.

Typically, the device must withstand the rigors of the chemical processing and serve as a suitable electrode in an electrolyte. Therefore, a passivating surface may be added to the semiconductor to make it more suitable to electrochemical processing. This film may be a thin film of SiO2, such as the native oxide of a silicon wafer or the native oxide developed onto a PECVD deposited amorphous silicon film, it may be TiO2, or it may be silicon nitride, silicon carbide, silicon oxynitride, silicon carbonitride, tetrahedryl amorphous carbon, or nitrogen doped tetrahedryl amorphous carbon or other suitable materials known in the art.

Ideally, the passivating film is itself semiconducting, because that enables the substrate fabrication to eliminate all patterning steps, as a thin chemically inert semiconductor would not contribute significant lateral electron currents or electrical impedance, especially if the semiconductor were itself a photoconductor. Alternatively, the top layer of the semiconductor may be crystallized [e.g., laser crystallization of amorphous silicon- Brendel et al. (2003), Thin Solid Films 427, Pages 86-90] to be more electrochemically inert. Alternatively, an extremely thin layer of a chemically inert metal (e.g., on the order of 2 nm), such as gold or one of the platinum group metals (including, but not limited to, PGM-ruthenium, rhodium, palladium, osmium, iridium, and platinum), known for their chemical inertness, can be deposited in such a manner as to prevent lateral currents. In an alternative embodiment, gold or a PGM can be patterned in an array of pads that are activated by the photoconducting substrate below. Alternatively, or additionally, a solution of nanoparticles or microparticles, such as, but not limited to, spin on glass, or a titania nanoparticle colloid, can be used to coat the substrate. In a further alternative, a monolayer of nanoparticles or microparticles can be self-assembled on the surface to create a uniform distribution of small electrically isolated pads.

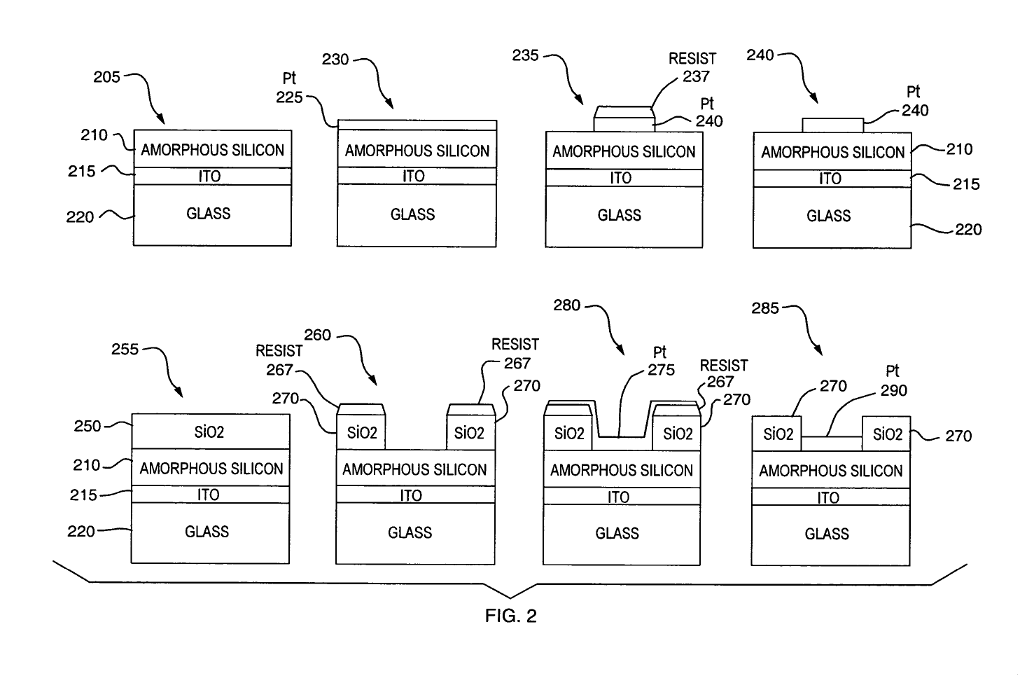

FIG. 2 depicts schematics of two embodiments of the process of photoelectrochemical placement of a material at a specific location on a substrate to create a photoelectrode substrate, according to one aspect of the present invention. In particular, FIG. 2 illustrates embodiments of the process steps for the patterning of platinum microcontacts on amorphous silicon. In FIG. 2, in a first embodiment, a device is fabricated with plasma enhanced vapor deposition 205 (PECVD) of amorphous silicon 210 onto conductive surface 215 on glass slide 220. In the embodiment shown, conductive surface 215 is indium tin oxide (ITO), but any suitable conducting surface may be used in the process of the invention. Next, a layer of platinum 225 (Pt) is deposited 230 on top of amorphous silicon 210. Platinum layer 225 is etched 235 using pattern resist 237 and HCL HNO3 at 70 degrees C. to create patterned platinum microcontact 240. Resist 237 is then stripped 245 with acetone, leaving patterned platinum microcontact 240.

In a second embodiment depicted in FIG. 2, patterned micro wells are created first. In FIG. 2, silicon oxide dielectric layer 250 (SiO2) is deposited 255 via PECVD onto amorphous silicon 210 on conductive surface 215 on glass slide 220. Silicon oxide layer 250 is etched 260 using pattern resists 267 and RIE, leaving patterned SiO2 270. Next, a layer of platinum 275 (Pt) is deposited 280 on top of amorphous silicon 210 and patterned SiO2 270. Resists 267 are then stripped 285 with acetone, NMP, and/or sonication, leaving patterned platinum microcontact 290. The finished device is then subjected to plasma-assisted oxidation 295. The fabrication may be made easier by eliminating the silicon oxide dielectric layer deposition, patterning, and etching steps if such a physical barrier between electrodes is not required.

In a specific demonstrative embodiment, the device is an ITO coated glass slide with a layer of intrinsic amorphous silicon and an array of platinum contacts coating the surface. The exposed silicon is oxidized thermally or electrochemically. The surface of the device is treated with colloidal silica particles and sintered under nitrogen, forming a porous matrix. The matrix is treated with triethoxysilanehydroxybutyramide. The device is treated with dimethoxytrityl chloride to protect free hydroxyls. The device is then treated with acetic anhydride to cap any unreacted nucleophiles on the surface. When in contact with an electrolyte solution of 10 mM hydroquinone and 50 mM tetrabutylammonium hexaflurophosphate in the fluidic (with a built-in counter electrode), the device is positively biased with respect to a platinum quasi-reference electrode to 1.7V, an operating region determined by viewing the redox signature of a known compound such as ferrocene. Light is projected through a digital micromirror array for 5 s (chopped at 10 Hz), after which the substrate is washed and then exposed to a cy3-phosphoramidite fluorescent dye, demonstrating the desired photoelectrochemical patterning.

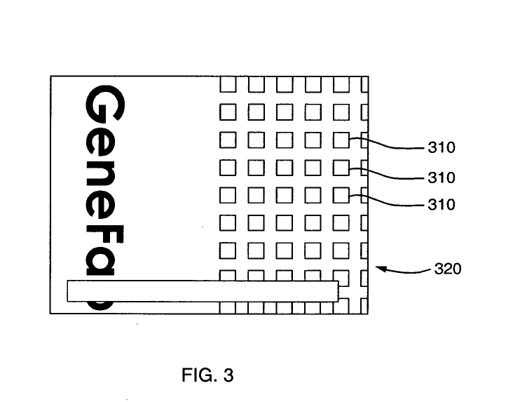

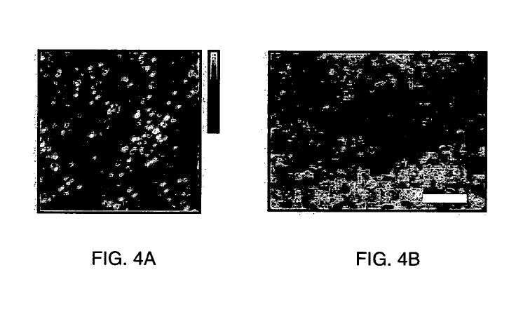

A scanning electron micrograph (SEM) of a device made according to the process of the present invention, having patterned platinum microcontacts 310 on amorphous silicon 320 over ITO-glass, is shown in FIG. 3. FIGS. 4A and 4B are Atomic Force Microscopy (AFM) and Scanning Electron Microscope (SEM) micrographs, respectively, of a suitable porous SiO2 reaction layer produced from sintered commercial colloidal silica, according to one aspect of the present invention.

Since electrochemistry will still occur at the semiconducting surface, the porous layer can serve as a high surface area platform for attachment of the desired chemistry, and surface integrity of the main substrate semiconductor is not as important. Alternatively, a semiconducting sol gel, such as TiO2 may be used to create a porous film and increase the surface area in contact with the electrolyte. Alternatively, instead of a semiconducting surface with which both electrochemical reactions and DNA synthesis occur, a photoelectrically active semiconducting solution, such as silver ion or semiconducting nanoparticles, can be flowed into the chamber. In these manners, solution impedance may be altered along the light path to allow for electrochemical reactions at the electrically active substrate surface, or for electrochemical reactions take place near the surface of the nano or micro particles in solution rather than on the surface, so the substrate may be glass or another material.

Illumination of the device can be with a diode array, digital micromirror array, LCD or LED screen or projection system, mirror-steered laser source, transillumination through a mask, laser-scanning, or any other light source that can be spatially modulated or temporally modulated with the device on a moveable stage. In a typical embodiment, a DMD array is used with a 1:1 image projection system, projecting a desired image on the substrate. The APO Rodagon D is a suitable lens for a simple 1:1 projection system, but other projection systems can utilize other types of relay lens systems, with or without magnification or reduction. The light source may be of any wavelength that suitably creates charge carriers in the semiconductor, i.e. wavelengths of light that exceed the bandgap either of the device or of the device with suitable chemical sensitizers.

It is desirable to generate acids, bases, or radicals at the surface in order to perform reactions of consequence. These reactions are typically spatially selective reactions that either promote or inhibit the deprotection or addition of monomers, oligomers, or pre-synthesized molecules, including the tandem use of photocleavable or photogenerated chemistries (e.g. the photoelectrochemical generation of a base to catalyze the photocleavage of a NPPOC group). Various reagents such as, but not limited to, water, hydroquinones, anthrohydroquinones, naphthohydroquinones, phenols, or other electrochemically active compounds capable of oxidation that yields a free proton may be used to generate acid at the surface in the pattern. A coupled electrochemical system may optionally be implemented, such as utilizing ferrocene as an electron mediator between the electrode and the acid/base generating compound. Typically this has advantages, in that the surface always reacts with the mediator in a known manner. Furthermore, the hydrolysis of water may also be used to generate an alkali or acid gradient at the surface with a different bias across the semiconductor. Additionally, electro-generated bases and radicals can be used in this device. The well-known need for generating such reactions in a specific pattern is a desired precondition for the production of arrays of oligonucleotides synthesized by the phosphoramidite method and arrays of peptides synthesized by standard peptide synthesis techniques.

It will be clear to one of ordinary skill in the art that other types of chemistries are possible with the present invention. With suitable substrate construction, solvents, and supporting electrolytes, almost all electrochemically controlled oxidation or reduction reactions are possible with this light addressable system. Photoelectrochemically-generated species can remove all acid, base, and radical cleavable protecting groups. Protecting groups may also be removed by direct electrochemical cleavage. This method is compatible with other biomolecule synthesis methods, including phosphotriester and H-phosphonate chemistries for DNA synthesis. Alternatively, the spatial photoelectrochemistry can be used to selectively functionalize various compounds onto the surface of the device, such as, but not limited to, proteins, DNA, and other types of biomolecules. Selective functionalization may be by acid-base, radical, by redox chemistry in solution, by redox chemistry directly on molecules linked to the solid support, or by redox chemistry applied during the coupling step to the desired compounds, including catalysis.

Once prepared, the device can selectively release the molecule from the substrate for the hierarchical assembly of larger constructs. Such hierarchical assembly schemes for genomic length DNA are disclosed in, for example, Carr, et al. (2005), U.S. Pat. App. Pub. No. US-2005-0255477 (“Method for High Fidelity Production of Long Nucleic Acid Molecules”). The molecule can be released using photoelectrochemically generated acids, bases, and radicals to cleave acid, base, or radical-labile linkers. Alternatively, photoelectrochemically-generated species can promote/inhibit the cleavage of molecules to be cleaved non-photoelectrochemically (i.e. chemically, bio-chemically, or by photocleavage) or vice-versa, including when the non-photoelectrochemical mechanism is either global or spatially localized (e.g. the photoelectrochemical generation of acid to inhibit the global deprotection of a succinyl linker by a base). Alternatively, the molecule can be released by the photocleavage of a photocleavable linker molecule if the photocleavage wavelengths are not present during the synthesis. In alternate embodiments, the molecule can be released by using a photogenerated acid, base, or radical with/without a photoelectrochemically generated inhibitor, and vice-versa, or the molecule can be released by creating a pH gradient that influences the interactions of biological molecules in both one-step (e.g. promoting/inhibiting nuclease activity) and multi-step schemes (e.g. affecting DNA hybridization that subsequently affects enzymatic cleavage).

In order to create high-density arrays with spot sizes on the order of 10 um by 10 um, it is necessary to inhibit lateral diffusion of the photoelectrochemically generated acid. Utilizing a high viscosity gel, matrix, or wax instead, or in addition to, a solvent solution is one approach. The electrolyte/matrix is applied in a solution phase and cooled or cured. After the desired reaction has occurred, the matrix is stripped with a solvent or by an increase in temperature. Another approach is to pattern micro wells by standard lithography techniques. In one embodiment, the patterned wells are made by depositing a dielectric material (such as silicon nitride or silicon dioxide) on the semiconductor via a spin on glass or chemical vapor deposition technique, applying and developing a photoresist, and etching the dielectric material with HF or reactive ion etching to expose the photoconductive layer at the sites desired. The wells prevent lateral diffusion.

Another approach to limit diffusion is the application of a microporous spin on glass or polymer capable of supporting phosphoramidite synthesis (i.e. resistance to multiple solvent rinses, oxidants, and electrochemical side reactions). Thus, synthesis occurs on the porous matrix rather than on the surface of the semiconducting device. Another method involves operating the device under an AC bias, which will generate acid and base under the appropriate bias. The AC- duty cycle can be altered to provide the desired quenching, thus inhibiting diffusion of protons. An alternate method involves altering the duty cycle of the light source, pulsing a specific spot and waiting for the acid to react in a given area before creating more acid. Another method involves altering both the duty cycle of the light source and power supply. In this case, a specific pattern of light is shown when the device is under one bias, and then an alternative pattern of light is shown when the device is under a different or reverse bias. Yet another method involves separating the non-bias electrode from the substrate surface by a distance less than the inter-photoelectrode distance on the substrate. In this manner, the counteracting chemicals generated at the non-bias electrodes will react with the reactive species before they diffuse between the photoelectrodes.

An alternate method involves the addition of chemical scavengers to the solution, such as pyridine, triethylamine, or any other suitable base (KOH, NaOH, etc.) for proton generation or extremely weak acids, such as ammonium chloride, to scavenge free bases. In the case of electrochemical diffusion for reaction on alternative surface, it may be necessary to use alternating current, such as a square wave, and illuminate the semiconductor at inverse locations as the current alternates. In this manner, acid and base can be generated at alternating spots so as to prevent lateral acid diffusion by an acid-base reaction. Additionally, in this case, a wire grid directly on the surface of the chip, in addition to any pads for desired electrochemistry necessary for combinatorial synthesis, may be connected to a global potential to generate a counteracting chemical to react with the diffusing species of interest. In this manner, diffusion of the species of interest reacts with the counteracting species before reaching any unintended surfaces.

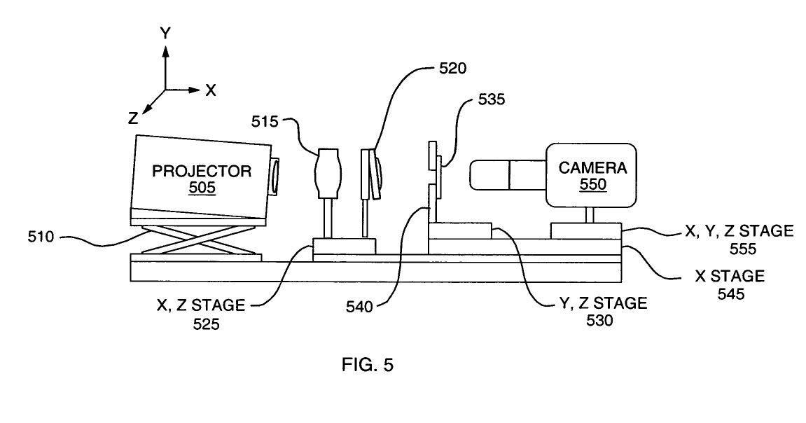

FIG. 5 is a schematic of an example embodiment of a DMD-based spatially modulated illumination system with spatial modulation capabilities for driving site-selective photoelectrochemistry, according to one aspect of the present invention. The system is low cost because it utilizes a commercially available digital light projector that contains its own light source. In FIG. 5, projector 505 is mounted on Y-Jack 510. APO Rhodagon D lens 515 and Gimble mount 520 are mounted on X,Z stage (1) 525, which controls zoom. Gimble mount 520 corrects for astigmatism. Y,Z Stage (3) 530 holds substrate 535 in vacuum chuck 540 and is mounted on X Stage (2) 545. Stage (2) 530 controls the focus of light onto substrate 535, while Stage (3) 530 controls the alignment of substrate electrodes to DMD pixels. Camera 550 is mounted on X,Y,Z Stage (4) 555, which is also mounted on x Stage (2) 545 and which controls camera focus and image scroll.

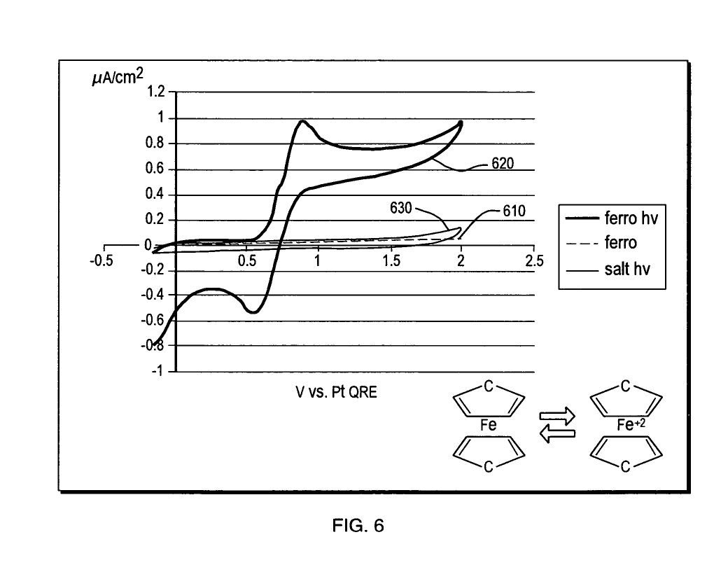

FIG. 6 is a photoelectrochemical cyclic voltammogram of ferrocene demonstrating selective photo-induced redox chemistry, according to one aspect of the present invention. Referring to FIG. 6 to demonstrate the selective photo-response of the substrate, cyclic voltammogram 610 was taken with 2 mM ferrocene and supporting electrolyte (50 mM tetrabutylammonium hexaflurophosphate) in acetonitrile, which clearly shows no current generated. Second cyclic voltammogram 620 is repeated during exposure to light (150 mW/cm2), clearly showing the CV signature of ferrocene. As a control, cyclic voltammogram 630 was taken during exposure to light with the salt solution only, in which case there was no electrochemical current generated. Thus, selective photo-induced redox chemistry is demonstrated. The fact that the non-illuminated curves do not resemble a typical ferrocene CV signature, but rather that of a resistor, indicates that the potential at the surface of the electrode is insufficient to drive electrochemistry because the potential drop of the semiconductor-electrolyte system is within the semiconductor. Furthermore, the small amount of current at zero and low biases when the substrate is illuminated indicates that the extent of photoelectrochemical reactions is insignificant compared to when the substrate is biased at or above its threshold potential. The CV curves of FIG. 6 also show that the photoconductor may be used as a light-sensitive electrochemical sensor, and thus the electrochemical generation of reagents can be monitored real-time in some embodiments.

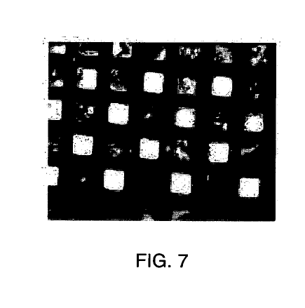

FIG. 7 is a fluorescence micrograph of a dye that has selectively reacted with functional groups that are only photoelectrochemically deprotected over illuminated electrodes, according to one aspect of the present invention.

The process of the present invention may optionally include real-time monitoring steps. These may include, but are not limited to, monitoring the deprotection reactions in real-time using UV absorption spectroscopy, monitoring the generation of deprotecting agents in real-time using a pH-sensitive dye, and electrochemically monitoring the generation of deprotecting agents using the photoelectrode.

The polymer arrays created by the method of the present invention have myriad uses. In one aspect of the present invention, a light-addressable potentiometric sensor is composed of an array created by the method of the present invention. In another aspect of the present invention, a photoconductor is used to create spatial pH gradients based on the spatial modulation of light in order to influence the interactions of biological molecules in a spatially selective manner. In one embodiment, the biological interaction is DNA hybridization. In another aspect of the present invention, a photoconductor is used to create spatial pH gradients based on the spatial modulation of light in order to influence the activity of enzymes in a spatially selective manner.

It is to be understood that the examples presented herein are illustrative of a broad range of other examples that may be constructed by combining steps involving the mechanisms detailed above. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications and substitutions by one of ordinary skill in the art are therefore also considered to be within the scope of the present invention, which is not to be limited except by the claims that follow.

What is claimed is:

1. A method for photoelectrochemical placement of a material at a specific location on a substrate, comprising the steps of:
providing a substrate having at the substrate surface at least one photoelectrode that is proximate to at least one molecule bearing at least one chemical functional group;
providing a reagent-generating chemistry co-localized with the chemical functional group and capable of generating reagents when subjected to a potential above a threshold; and
applying an input potential to the photoelectrode to create a patterned substrate, the input potential exceeding the threshold in the presence of light and not exceeding the threshold in the absence of light.

2. The method of claim 1, wherein the chemical functional group is protected and the generated reagents are deprotecting.

3. The method of claim 1, wherein the chemical functional group is unprotected and the generated reagents are activating.

4. The method of claim 2, wherein the protected chemical functional group is located on a second parallel substrate and the reagent-generating chemistry can diffuse towards the protected chemical function group on the second substrate.

5. The method of claim 1, wherein the generated reagents promote the removal of a protecting group from the chemical functional group by another agent.

6. The method of claim 1, wherein the generated reagents promote the addition of a monomer.

7. The method of claim 1, wherein the generated reagents inhibit the removal of a protecting group from the chemical functional group by another agent.

8. The method of claim 1, wherein the generated reagents inhibit the addition of a monomer.

9. The method of claim 1, in which the photoelectrode is selected from the group consisting of a semiconductor, silicon, an organic photoconductor, titanium dioxide, dye sensitized titanium dioxide, a schottky diode, a layered structure of silicon and another semiconductor, a P-I-N diode, a P-N junction, and a P-N junction having a top layer coated with an inert metal.

10. The method of claim 1, wherein the applied input potential is selected from the group consisting of: the peak potential of the substrate, within +/−0.5V of the peak potential of the substrate, AC, AC and synchronized with the illumination source, pulsed, and pulsed and synchronized with the illumination source.

11. The method of claim 1, wherein the photoelectrode acts as a photoconductor that generates a potential that is approximately linearly proportional to an applied light field and is biased below the threshold.

12. The method of claim 1, wherein the photoelectrode acts as a photoconductor that is biased above the bandgap threshold potential of the substrate so that there exists sufficient energy for the electrons to overcome the bandgap when no light is applied.

13. The method of claim 1, further comprising the step of generating getters for rendering neutral the generated reagents by oppositely biasing, to the photoelectrode, one or more adjacent photoelectrodes.

14. The method of claim 13, wherein the getters spatially localize the effect of any deprotecting reagents.

15. The method of claim 14, wherein the deprotecting reagents are acids and the getters are bases, the deprotecting reagents are acids and the getters are radicals, the deprotecting reagents are bases and the getters are acids, the deprotecting reagents are radicals and the getters are acids, or the deprotecting reagents are radicals and the getters are radicals.

16. An array comprising at least one region of a material specifically placed by the method of claim 1.

17. A light-addressable potentiometric sensor comprising an array created by the method of claim 16.

18. The method of claim 1, further comprising the step of providing a porous reaction layer disposed on the substrate.

19. The method of claim 1, wherein the source of the light is a spatially addressable light source.

20. The method of claim 19, wherein the spatially addressable light source consists of one or more elements selected from the group consisting of: digital mirror devices, OLED display, frontlit LCD display, backlit LCD display, fluorescent LCD display, LED display, and scanning light source display.

21. The method of claim 19, wherein the spatially addressable light source is temporally modulated.

22. The method of claim 1, wherein the photoelectrode is a continuous photoelectrode such that different regions of the photoelectrode may be differentially optically addressed.

23. The method of claim 22, further comprising the step of differentially optically addressing the continuous photoelectrode to create a spatial pattern of material.

24. The method of claim 1, wherein there is a one-dimensional or 2-dimensional array of photoelectrodes.

25. The method of claim 24, wherein the array of photoelectrodes are differentially optically addressed to create a spatial pattern of material.

26. The method of claim 2, further comprising the step of monitoring the generation of deprotecting agents in real-time using a pH-sensitive dye.

27. The method of claim 2, further comprising the step of monitoring the deprotection reactions in real-time using UV absorption spectroscopy.

28. The method of claim 2, further comprising the step of electrochemically monitoring the generation of deprotecting agents using the photoelectrode.

29. A method for photoelectrochemical synthesis of a polymer array, comprising the steps of:
(a) providing a substrate having at the substrate surface at least one photoelectrode that is proximate to at least one molecule bearing at least one chemical functional group;
(b) providing a reagent-generating chemistry co-localized with the chemical functional group capable of generating reagents when subjected to a potential above a threshold;
(c) applying an input potential to the photoelectrode to create a patterned substrate, the input potential exceeding the threshold in the presence of light and not exceeding the threshold in the absence of light; and
(d) repeating steps (a) to (c) until a polymer array of desired size is synthesized.

30. The method of claim 29, further comprising the step of immobilizing pre-synthesized molecules into patterns and arrays.

31. A polymer array produced by the method of claim 29.

32. A DNA array produced by the method of claim 29.

33. A method for photoelectrochemical placement of a material at a specific location on a substrate, comprising the steps of:
providing a substrate having at the substrate surface at least one photoelectrode that is proximate to at least one molecule bearing at least one chemical functional group that can be cleaved;
providing a cleaving reagent-generating chemistry co-localized with the chemical functional group that is capable of generating cleaving reagents when subjected to a potential above a threshold; and
applying an input potential to the photoelectrode to create a patterned substrate, the input potential exceeding the threshold in the presence of light and not exceeding the threshold in the absence of light.

34. The method of claim 33, wherein the cleaving agent selectively promotes the cleavage of a molecule from a surface by another agent.

35. The method of claim 33, wherein the cleaving agent selectively inhibits the cleavage of a molecule from a surface by another agent.
